(19) United States
(12) Patent Application Publication
(10) Pub. No.: US 2010/0145377 A1
(43) Pub. Date: Jun. 10, 2010

(54) Lancing Device For Minimizing Pain

(75) Inventors: Siew Kong LAI, Skudai (MY); Chow Kian Yeo, Singapore (SG)

Correspondence Address: LAWRENCE Y.D. HO & ASSOCIATES PTE LTD, 30 BIDEFORD ROAD, #02-02, THONGSIA BUILDING, SINGAPORE, 229922, SG

(73) Assignee: Venture Corporation Limited, Singapore (SG)

(21) Appl. No.: 12/327,817

(22) Filed: Dec. 4, 2008

(51) Int. Cl. A61B 17/34

(52) U.S. Cl. 606/182; 606/181

(57) ABSTRACT

The present invention provides a lancing device designed for minimizing pain to the user, comprising a probe with cam profile responsible for controlling the lancet speed such that the lancet enters the skin relatively fast, decelerates smoothly and gradually to zero velocity at maximum depth of penetration and retracts slowly and controllably. The housing of the lancet comprises of leaf springs and set of guides for minimizing or eliminating pitching or vibration of the lancet during lancing, hence reducing the pain experienced by the user. The lancing device is further equipped with a gear-damper system for minimizing or eliminating noise produced during lancing process.

### FIELD OF THE INVENTION

The present invention generally relates to a lancing device, and in particular to a lancing device for minimizing pain to the user while withdrawing sample fluid.

### BACKGROUND OF THE INVENTION

Lancing device, or also called a lancet device, is typically used in the medical field to lance or break the surface of the skin of one's finger, in order to extract a small blood sample for self diagnostic purposes.

Good diabetes management requires frequent self-monitoring of blood glucose level through self-testing. Lancing device is a critical tool for obtaining blood samples for glucose measurement. Self-testing of blood glucose is important, as it enables people with diabetes to know their blood glucose level at any time, hence allowing them to exercise tighter blood glucose control. This will help to prevent any potentially serious consequences of very high or very low blood glucose level. It is especially crucial for people who take insulin, as self-testing will allow more accurate dosage adjustment.

The primary mechanism of most lancing devices currently existing in the market, both for repeated use and disposable lancet types, involved the priming of a spring-based system, followed by a release of a trigger to launch the lancet or needle into the finger of the user. In this way, the lancet or needle is made to puncture a tiny hole on the finger of the user, for a blood sample to be extracted for diagnostic purposes.

Such lancing devices generally convert the potential energy from the primed spring into the kinetic energy of a moving lancet and its holder at the same time. This kinetic energy is then dissipated through the impact of the lancet and its holder against a rigid stop, which is also often used as a way of defining the depth of penetration of the needle into the user's finger. In most cases, the residual kinetic energy is used, to reverse the motion of the lancet, and hence extracting it from the finger after puncturing a hole in it.

It is quite typical to hear complaints from users of the lancing devices with design described above, in relation to pain during lancing process. This could be attributed to some of the following reasons. The lancing mechanism hitting at a hard stop at maximum velocity would cause excessive impact vibration, which will then be transmitted to the lancet. The excessive relative vibration and movement between needle and finger is likely the cause of the pain experienced by user.

Another cause of pain during lancing is an uncontrolled lancing motion of the lancet, which will result in an unpredictable trajectory of the needle during lancing process. This uncontrolled motion refers to the ability of the lancet and its holder to move within the sliding clearance offered by its guides, which are often plastic molded features. In addition to that, impact noise is perceived as pain most of the time, since it forms part of the overall user experience. Devices with such lancing mechanism, which relies on impact to define the lancet's penetration depth and to reverse its motion, are often perceived by the user as being noisy and painful.

Examples of lancet device with a design intended to allow less painful blood withdrawal, may be seen in the following U.S. Patents. U.S. Pat. No. 4,924,879 discloses a blood lancet device, which convert the relaxation movement of the drive spring by means of a rotatable drive rotor into the prick movement, hence allowing blood withdrawal with little or no pain. The vibration caused by the impact of the lancet holder onto a hard stop can then be avoided. The rotor is driven by a coaxial coil spring and the rotation movement of the rotor is converted to the linear movement of the lancet by means of a push rod system.

U.S. Pat. No. 5,318,584 discloses a lancing device with the drive rotor having a rotation axis parallel to the prick direction and is also driven by a coaxial coil spring. The conversion of the rotational movement into the necessary linear movement of the lancet holder is performed by a rotary drive. The design allows a very good pricking behavior with low vibrations and a reproducible pricking depth, hence resulting in less pain. U.S. Pat. No. 4,203,446 discloses a spring lancet holder with improved accuracy and reproducibility of puncture wounds in the skins by minimizing the recoil transmitted to the lancet holder by actuation of the drive mechanism, which pushes the lancet into the skin.

However, these prior arts only reduced the extent of the above mentioned problem, as the basic working principle and mechanism of the lancing device remain unchanged, by making use of hard stop to stop an advancing lancet to achieve depth of penetration, resulting in kinetic energy being dissipated through impact.

### SUMMARY OF THE INVENTION

The following presents a simplified summary to provide a basic understanding of the present invention. This summary is not an extensive overview of the invention and is not intended to identify key features of the invention. Rather, it is to present some of the inventive concepts of this invention in a generalized form as a prelude to the detailed description that is to follow.

The present invention provides a lancing device for minimizing pain to the user while withdrawing sample fluid. According to one aspect of the invention, there is provided a lancing device comprises of a lancet, a housing comprising leaf springs and guides for minimizing or eliminating pitching or vibration of the lancet during lancing, a probe with cam profile responsible for controlling the lancet speed such that the lancet enters the skin relatively fast, decelerates smoothly and gradually to zero velocity at maximum depth of penetration and retracts slowly and controllably, a gear-damper system for minimizing or eliminating noise produced during lancing process, and a priming system.

In one embodiment of the present invention, the guides comprise of v-shaped guides and profile guides, which are used together with the leaf springs for minimizing or eliminating probe movement in x- and y-axis during lancing process.

In another embodiment of the present invention, the probe with cam profile comprises of profile slides having its rotation centre coinciding with the lancet's rotation centre.

In yet another embodiment of the present invention, the probe with cam profile comprises of slotted guides for limiting rotational movement of the probe during lancing process.

In another embodiment of the present invention, the priming system comprises of a torsion spring, a probe actuator, a priming gear, a rack, a priming button, a compression spring and a fire button.

In yet another embodiment of the present invention, the probe actuator comprises of an integrated damper and a cam follower.

The objectives and advantages of the present invention will become apparent from the following detailed description of embodiments thereof in connection with the accompanying drawings.

### BRIEF DESCRIPTION OF THE DRAWINGS

Preferred embodiments according to the present invention will now be described with reference to the figures accompanied herein, in which like reference numerals denote like elements.

FIG. 1 is a perspective view of a lancing device according to one embodiment of the present invention;

FIG. 2 is a top perspective view of a top case of the lancing device in accordance with one embodiment of the present invention;

FIG. 3 is an exploded bottom view of the top case of the lancing device in accordance with one embodiment of the present invention;

FIG. 4 is an exploded view of a bottom case of the lancing device in accordance with one embodiment of the present invention;

FIG. 5 is a top perspective view of a probe with cam profile of the lancing device in accordance with one embodiment of the present invention;

FIG. 6 is a bottom perspective view of the probe with cam profile of the lancing device in accordance with one embodiment of the present invention;

FIGS. 7 a-b show lancet displacement profiles in z-x and z-y axes of the lancing device in accordance with one embodiment of the present invention and its competitor;

FIG. 8 is a top perspective view of a probe actuator of the lancing device in accordance with one embodiment of the present invention;

FIG. 9 is a bottom perspective view of the probe actuator of the lancing device in accordance with one embodiment of the present invention;

FIG. 10 is a cross sectional view of a connection between the probe actuator and a bottom case of the lancing device in accordance with one embodiment of the present invention;

FIG. 11 is a top perspective view of a final assembly of the lancing device in accordance with one embodiment of the present invention.

### DETAILED DESCRIPTION OF THE INVENTION

Description of certain embodiments of the present invention shall now be explained in detail, with reference to the attached drawings. It is to be understood that no limitation of the scope of the invention is thereby intended, such alterations and further modifications in the illustrated device, and such further applications of the principles of the invention as illustrated therein being contemplated as would normally occur to one skilled in the art to which the invention relates.

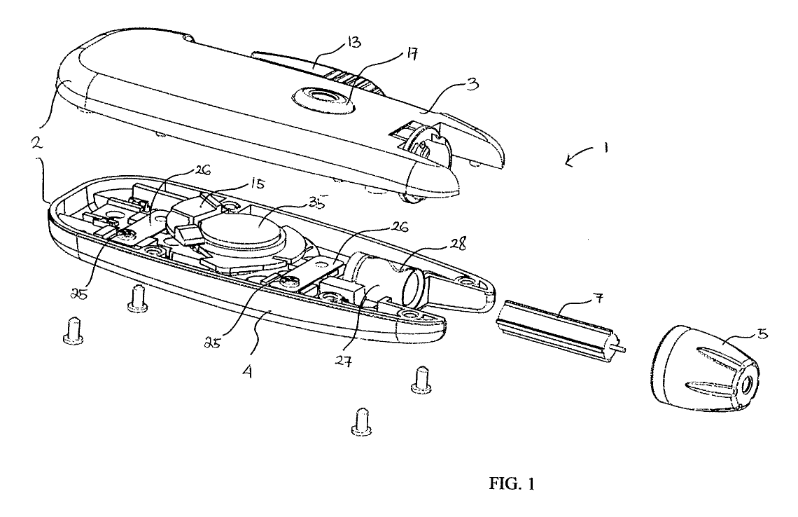

FIG. 1 shows a perspective view of a lancing device according to one embodiment of the present invention. In this embodiment of the present invention, a housing 2 of the lancet device 1 comprises of top case 3 and bottom case 4. Screws are used to hold the top case 3 and bottom case 4 together. In another embodiment of the present invention, the top case 3 and bottom case 4 are joined together by ultrasonic welding. A cap 5 is disposed at the front end of the housing 2, which has an opening 6 for exit and reentry of the lancet 7. The top 3 and bottom case 4 follows an oval shape for easy holding.

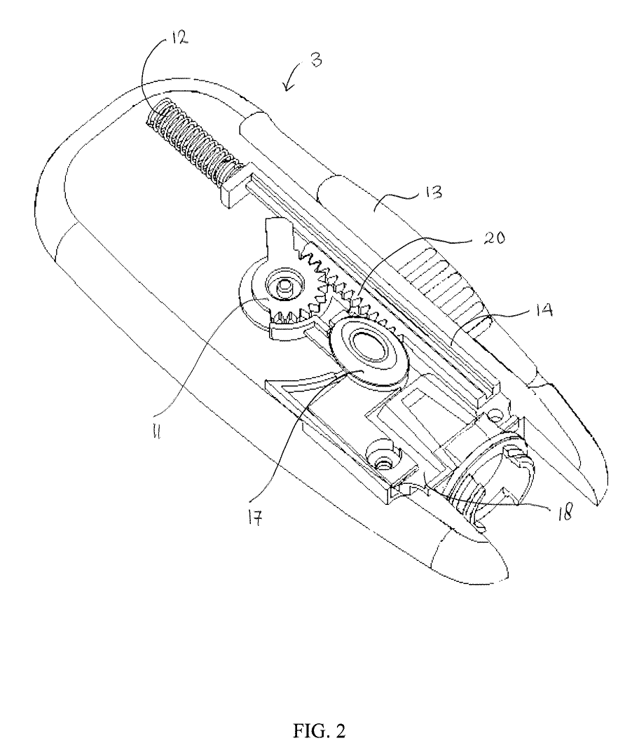

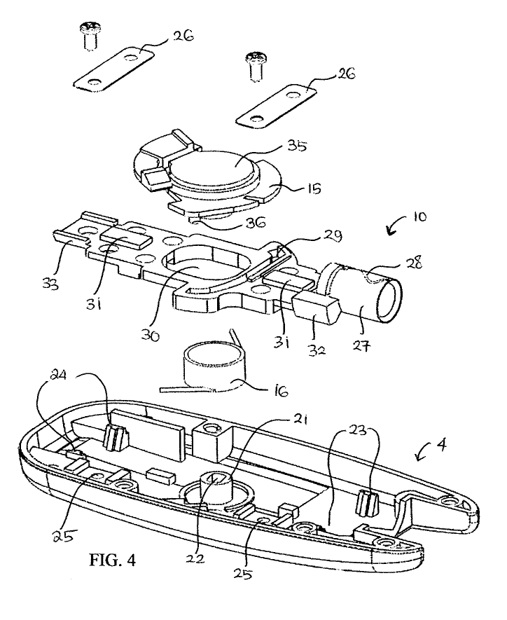

FIG. 2 shows a top perspective view of the layout of the top case assembly of the lancet device according to one embodiment of the present invention. A priming system employed in this embodiment comprises of a priming gear 11, a compression spring 12, a priming button 13, a rack 14, a probe actuator 15, a torsion spring 16 and a fire button 17. Details of the configuration of the priming system are further illustrated in FIG. 4, which shows an exploded bottom view of the top case of the lancing device according to one embodiment of the present invention. A case top cover 18 is needed to connect the priming gear 11 and the fire button 17 to the top case 3 via screws connection. The priming gear 11 is coupled to the rack 14 in such a way that the teeth 19 of the priming gear engage the teeth 20 of the rack.

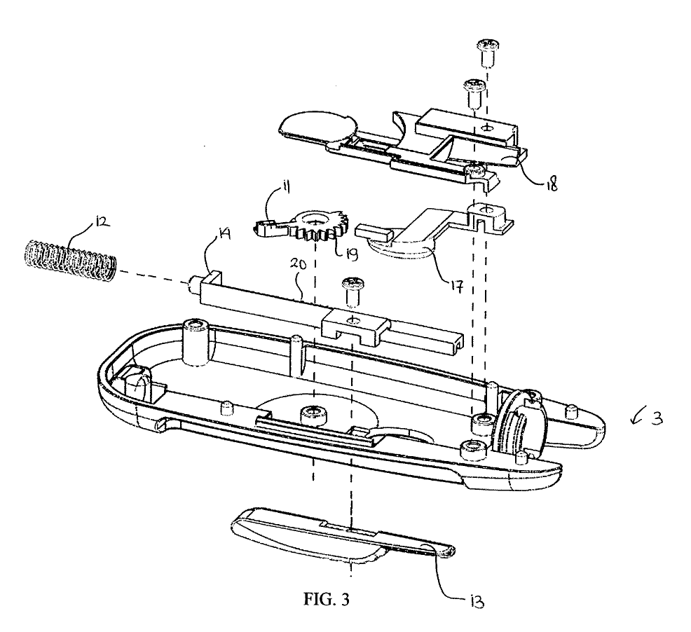

The priming button 13 is connected to the rack 14 via screws connection, as illustrated in FIG. 3. When the lancet user pulls the priming button 13 downward, the rack 14 is then driven down to a stop position, thereby causing the priming gear 11 to rotate in counter-clockwise direction. The priming gear 11 is coupled to the probe actuator, such that the rotational motion applied to the priming gear 11 will then be used to prime the probe actuator 15. The probe actuator 15, together with the torsion spring 16 where the potential energy is stored, are then locked in the primed position and will only be released once the fire button 17 is pressed. Once the fire button 17 is pressed, the stored potential energy from the torsion spring 16 will then be imparted to the probe actuator 15, resulting in the forward sliding of the probe 10 together with the lancet 7 towards the skin of the user. The compression spring 12 serves the function for returning the priming button 13 to the original position.

Details of the layout of the bottom case assembly of the lancet device according to one embodiment of the invention are illustrated in FIG. 4. The bottom case 4 also has a guide pin 21 projected out from the middle of the case. The guide pin 21 has a frame slot 22 for receiving and coupling with the probe actuator 15. Two v-shaped profile guides 23 and 24 are disposed on the front and rear part of the bottom case 4 for locating a probe with cam profile 10. There are also two pin connections 25 located on one longer side surface 8 of the bottom case, for attachment of two leaf springs 26. The leaf springs 26 are used for suspending the probe with cam profile 10 in the lancing device 1, such that the movement of the probe 10 in z-axis is eliminated during sliding of the lancet 7. In other words, the leaf springs 26 are used to ensure that the probe with cam profile 10 will always be in contact with the v-shaped profile guides 23 and 24 of the bottom case 4. This is done to minimize pitching of the lancet 7 during lancing, hence reducing the pain.

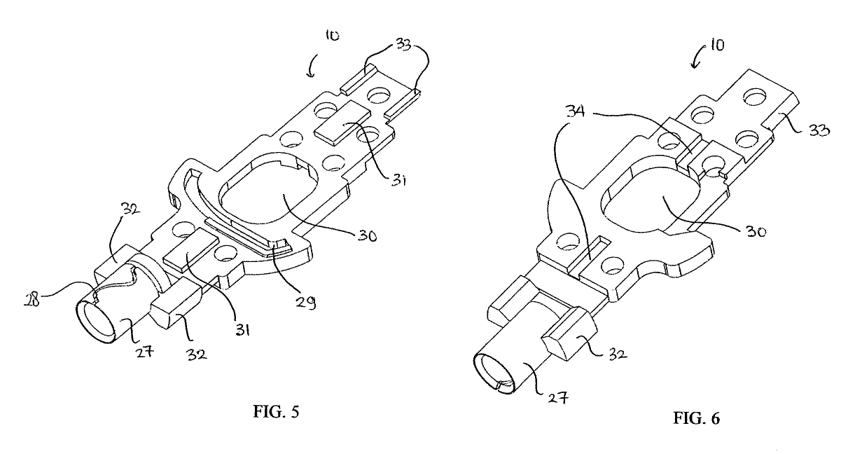

FIG. 5 illustrates a perspective top view of the probe with cam profile 10 according to one embodiment of the present invention. There is an integrated lancet collar 27 disposed at the front part of the probe 10. The lancet collar 27 has a cutaway 28 for providing a gripping force to the lancet 7. The cutaway 28 offers the flexibility to open up the lancet collar 27 after the lancet 7 is inserted. Hence, the lancet 7 can be easily removed and replaced accordingly. The probe 10 has an embedded cam profile 29 and a slotted opening 30 in the middle part for receiving the probe actuator 15 there through. Two raised pads 31 for contacting leaf springs 26 are positioned before and after the slotted opening 30 respectively. The probe 10 is further equipped with front 32 and rear 33 profile slides. The contact surfaces of profile slides 32 and 33 have circular profiles whose center axes coincide with that of the lancet center. This will limit the probe movement, if any, to a slight minimum rotation of the lancet, instead of lateral movement of the lancet, thus reducing any pain experienced by the user to a minimum. The front profile slides 23 are slightly larger than the rear profile slides 24. FIG. 6 shows a perspective bottom view of the probe with cam profile 10 according to one embodiment of the present invention. There are slotted guides 34 in the front and rear part of the probe 10 for limiting the rotational movement of the probe during lancing process.

Referring to FIG. 4 and FIG. 5, the cam profile 29 is molded into the probe 10 and is responsible for regulating the speed of the lancet 7. The velocity profile of the lancet 7 is controlled by the cam system. In other words, contouring the cam profile 29 would allow the related lancet displacement and velocity profile to be optimized for minimum pain and enhanced user compliance. The lancet 7 penetrates the skin relatively fast but decelerates smoothly and gradually to zero velocity at the maximum depth of penetration into the target area, where the nerve endings are abundant. The smooth transition to zero velocity and absence of vibration reduces pain to the user. Slow and controlled retraction of the lancet will prevent the wound channel to collapse and allow the blood to flow directly to the surface. This feature encourages rapid healing of the puncture wound and offers less painful lancing experience to the user at the same time.

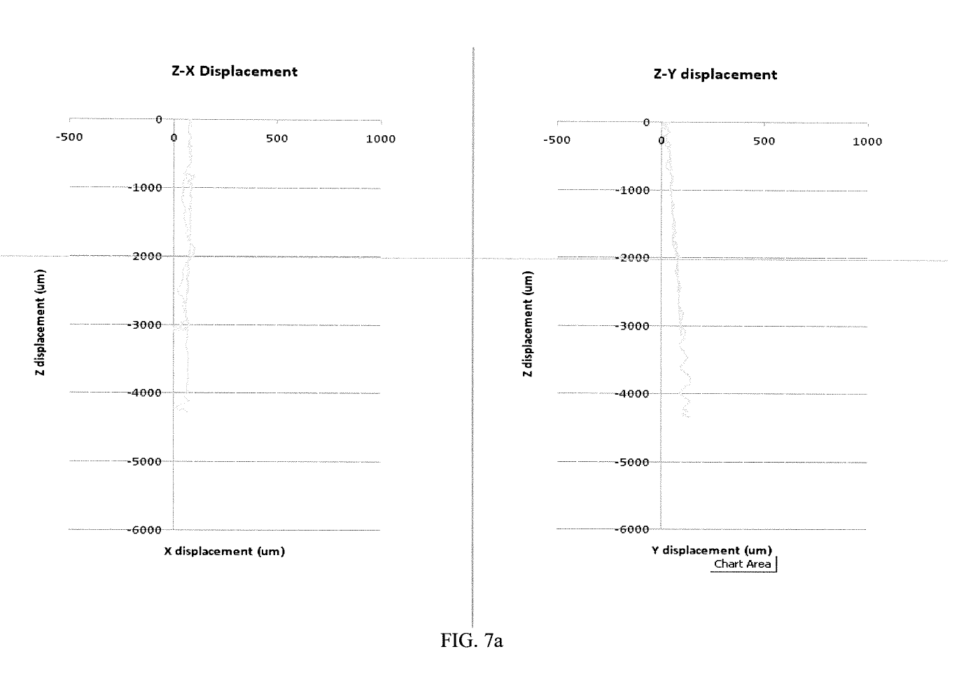

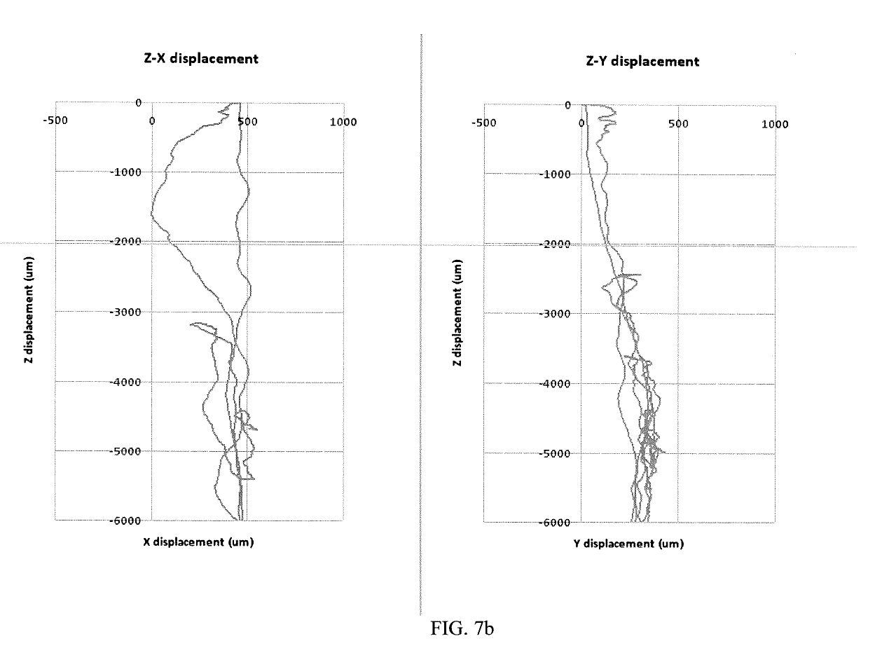

FIGS. 7 a-b show the comparison of the lancet displacement in z-x and z-y axes during the lancing process, between the lancet device of the present invention (FIG. 7a) and a leading lancet product (FIG. 7b). The displacement profiles clearly show that there is minimum or no lateral movement of the lancet device of the present invention during the lancing process. The lancet of the present invention enhances controlled motion of the lancet for its entry and withdrawal from the skin of the user during the lancing process, as shown by an almost straight line in its displacement profiles indicating little or no lateral movement of the lancet during its entire lancing trajectory. This feature allows the pain experienced by the user during lancing process to be reduced to the minimum level and serves as a significant improvement to the competitor's product.

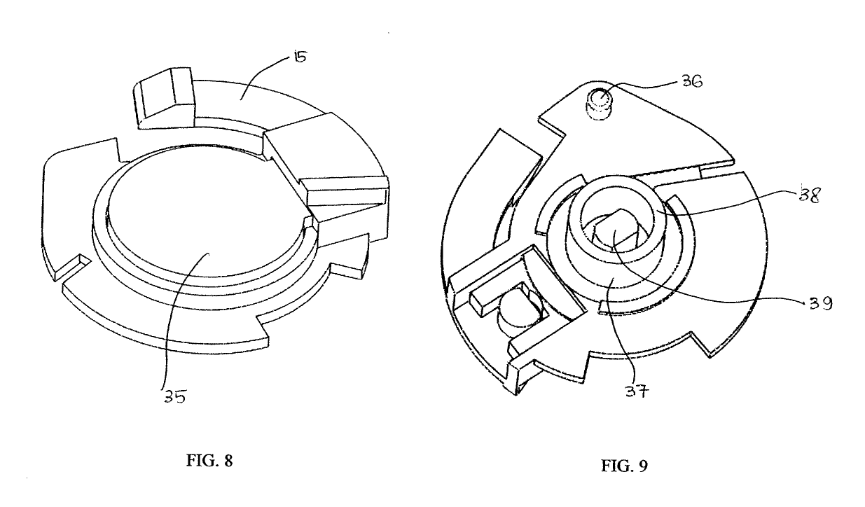

FIG. 8 and FIG. 9 illustrate a perspective top and bottom view of the probe actuator of the lancing device respectively, according to one embodiment of the present invention. The probe actuator 15 is equipped with an integrated damper 35 positioned in the middle part, such that the damper 35 can be coupled to both the priming gear 11 and the probe actuator 15 at the same time. The probe actuator 15 is further equipped with a cam follower 36, which drags over the cam profile surface 29, accurately tracing the surface of the cam. A frame slot 37 is located in the middle of the probe actuator 15 and comprises of two different parts; an outer circular ring 38 for capping over the guide pin 21 of the bottom case 4 and a small protrusion 39 from the damper 35, located inside the circular ring 38 to be inserted to the frame slot 22 of the guide pin 21 of the bottom case 4.

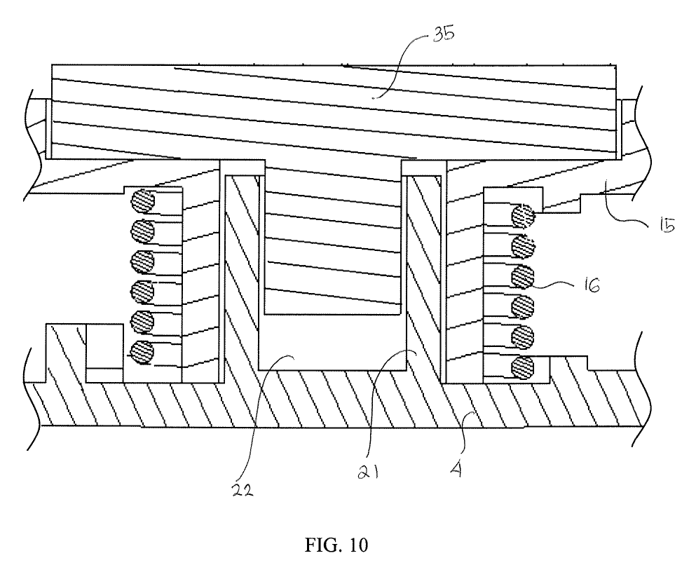

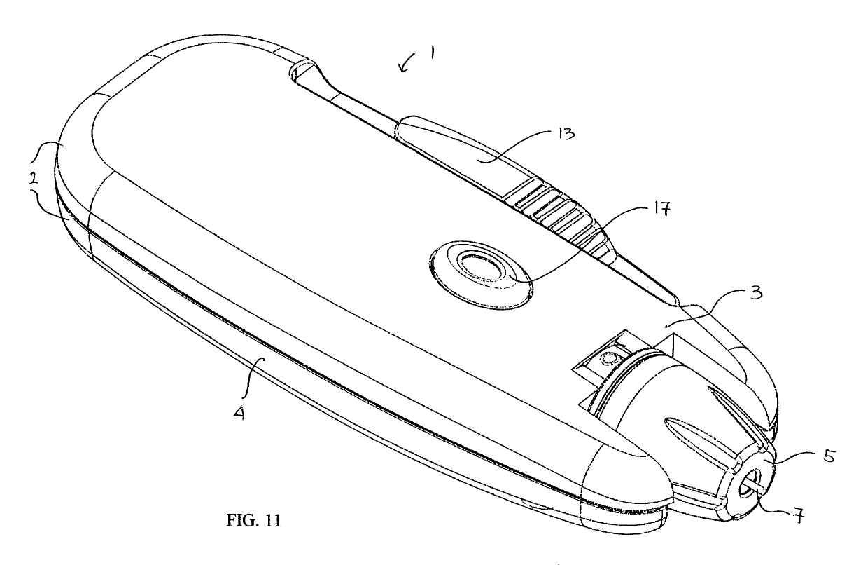

The connection between the damper 35, the probe actuator 15, torsion spring 16 and guide pin 21 of the bottom case 4 is further illustrated in FIG. 10. The inner surface of the guide pin 21 interacts with the damper 35, whereas the outer surface of the guide pin 21 interfaces with the circular ring 38 of the probe actuator 15, providing guidance for the probe 10. The torsion spring 16 is positioned to be resting on outer surface of the probe actuator 15. Hence, the kinetic energy of the propelling lancet of the present invention is not dissipated through impact but rather through a gear-damper system. The gear-damper configuration will minimize or even eliminate the noise produced during the lancing process and will enhance the user's compliance significantly.

Lancing device 1 according to the preferred embodiment in the present invention is designed such that any horizontal or vertical translation movement is minimized and the only freedom of movement allowed is rotation of the probe 10. The clearance between the slotted guides 34 and the guide pin 21 is the one determine the extent of the rotation. The profile slides 32 and 33 sit on the v-shaped profile guides 23 and 24, rotating with the same center of rotation as that of the lancet 7, while the probe 10 sliding forward and backward during lancing process. The v-shaped profile guides 23 and 24 are meant to eliminate movement of the probe 10 in y-axis during sliding of the lancet 7. The design of the lancet device 1, is intended to minimize or eliminate the pitching and vibration of the lancet 7 and its holder, during lancing. This means that the lancet 7 is always guided, without any sliding clearance for freedom of motion in horizontal or vertical direction, during its entire lancing trajectory. The only allowed freedom of motion for the lancet 7 during sliding is rotation, while horizontal or vertical translation is minimized or eliminated. The coincidence of the rotation centre of the lancet 7 and the profile slides 32 and 33, will ensure minimum or no lateral and only limited rotational movement of the lancet 7 during lancing process, and hence minimizing wound trauma and prevent the collapse of the wound channel, which will significantly reduce the pain experienced by the user during lancing.

Besides cam profile 29, the ratio of the damper 35 and the stiffness of the torsion spring 16 are other factors that determine the velocity profile of the lancet 7 It is preferred if the torsion spring 16 is not too stiff, as it will require more efforts from the user to prime it. The use of less stiff spring is compensated by proportionally reducing the damping. The damping effect can be appropriately adjusted by using different size of damper. Lastly, the cam profile 29 will determine how much of the potential energy from the torsion spring 16 is converted to the kinetic energy of the lancet 7. In summary, the combination of the effect of different cam profile 29, different stiffness of the torsion spring 16 and different ratio of the damper 35 can be optimized for achieving desired velocity profile of the lancet 7.

After the lancing process, the lancet is then retracted from the skin of the user and the probe 10 will slide backward as the cam follower 36 of the probe actuator 15 moves along the cam profile 29 embedded in the probe 10. A top perspective view of the final assembly of the lancet device according to one embodiment of the present invention is shown in FIG. 12.

1. A lancing device, comprising:
a lancet;
a housing comprising guides for minimizing pitching of the lancet during lancing;
a probe with cam profile for controlling the speed of the lancet such that the lancet enters the skin quickly, decelerates at standstill at maximum depth of penetration and retracts controllably;
a gear-damper system for minimizing noise produced during lancing process; and
a priming system.

2. The lancing device as claimed in claim 1, wherein said guides comprise of v-shaped guides and profile guides, said v-shaped guides and profile guides are used together with leaf springs for minimizing or eliminating probe movement in x- and y-axis during lancing process.

3. The lancing device as claimed in claim 1, wherein said probe with cam profile comprises of profile slides having its rotation centre coinciding with the lancet's rotation centre.

4. The lancing device as claimed in claim 1, wherein said probe with cam profile comprises of slotted guides for limiting rotational movement of the probe during lancing process.

5. The lancing device as claimed in claim 1, wherein said priming system comprises of a torsion spring, a probe actuator, a priming gear, a rack, a priming button, a compression spring and a fire button.

6. The lancing device as claimed in claim 5, wherein said probe actuator comprises of an integrated damper and a cam follower.
